(19) United States
(12) Patent Application Publication
(10) Pub. No.: US 2025/0120359 A1
(43) Pub. Date: Apr. 17, 2025

(54) A TOMATO PLANT COMPRISING DOMINANT RESISTANCE GENES TO TOMATO BROWN RUGOSE FRUIT VIRUS

(71) Applicants: PHILOSEED LTD, Hazav (IL); NRGENE TECHNOLOGIES LTD, Ness Ziona (IL)

(72) Inventors: Irit Gilan, Kfar Harif (IL); Ehud Kopeliovitch, Mazkeret Batya (IL); Ben Gradus, Ness Ziona (IL); Doron Cohen, Ness Ziona (IL); Barak Markus, Ness Ziona (IL); Arie Zackay, Ness Ziona (IL); Yedael Waldman, Ness Ziona (IL)

(21) Appl. No.: 18/730,751

(22) PCT Filed: Jan. 30, 2023
(86) PCT No.: PCT/IL2023/050102

§ 371 Date: Jul. 19, 2024

Related U.S. Application Data
(60) Provisional application No. 63/304,644, filed on Jan. 30, 2022.
(60) Provisional application No. 63/358,156, filed on Jul. 4, 2022.

(51) Int. Cl. A01H 1/00; A01H 1/04; C12Q 1/6895

(52) U.S. Cl. CPC A01H 1/126; A01H 1/04; C12Q 1/6895; C12Q 2600/13; C12Q 2600/156

(57) ABSTRACT

The present invention relates to the field of agriculture, more particularly to tomato plants harboring dominant resistance to the recently characterized Tobamovirus—Tomato Brown Rugose Fruit Virus. Tobamoviruses are a genus of plant viruses which pose a significant threat to vegetable and ornamental crops around the world. The impact of these viruses on crops can be economically devastating and result in considerable yield losses. The present invention discloses a tomato plant harboring a partially dominant resistance gene/genes to tomato brown rugose fruit virus (ToBRFV). It is another object of the present invention to disclose genetic markers for the use in identification and/or selection of tomato plants harboring partially dominant resistance to tomato brown rugose fruit virus (ToBRFV)

### FIELD OF THE INVENTION

The present disclosure relates to the field of agriculture, more particularly to tomato plants harboring dominant resistance to the recently characterized Tobamovirus—Tomato Brown Rugose Fruit Virus.

### BACKGROUND OF THE INVENTION

Tobamoviruses are a genus of plant viruses which pose a significant threat to vegetable and ornamental crops around the world, especially plants belonging to the Brassicaceae, Cucurbitaceae, Solanaceae and Malvaceae families. The impact of these viruses on crops can be economically devastating and result in considerable yield losses. Among these viruses are viruses which adversely infect tomato plants, the main ones being the tobacco mosaic virus (TMV), tomato mosaic virus (ToMV) tomato mild mottle virus (ToMMV), tobacco mild green mosaic virus (TMGMV) and pepper mild mottle virus (PMMoV).

Tobamovirus genus is the largest genus (comprising 35 species) among the seven genera in the Virgaviridae family. These viruses are characterized by having a 300 nm long rod-shaped particle encapsulating a single-stranded, positive sense RNA genome of 6.2 to 6.4 kb encoding four proteins: the genomic segment expresses two replication-related proteins of 126 and 183 kDa, resulting from partial suppression of a stop codon; a 30-kDa movement protein (MP) is expressed through a sub-genomic RNA1 (sgRNA1); and a 17.5-kDa coat protein (CP) is expressed from a second sub-genomic RNA2 (sgRNA2).

In the spring of 2015 the occurrence of a new tobamovirus in tomato (Solanum lycopersicum, cv. Candela) was discovered in greenhouses in Jordan (Salem et al., 2016). The newly discovered virus was not shown to phylogenetically align with either ToMV or the TMV clades, but to stem from a branch leading to the TMV clade. The symptoms of the new virus included mild foliar symptoms at the end of the season, but strong brown rugose symptoms on the fruits. A later publication showed that the virus was also present in Israel in 2014, and it was established that the virus can also infect pepper (Capsicum annuum) plants (Luria et al., 2017).

Additionally, it was demonstrated that tomato plants harboring known resistance genes to tobamoviruses exhibited susceptibility to the new virus (the virus was observed to break the resistance of the commonly used resistance genes against ToMV: Tm-1, Tm-2, and Tm-2²). As the virus was clearly different from other known tobamoviruses, it was given a new designation: Tomato brown rugose fruit virus (ToBRFV).

Symptoms appear to vary based on the affected variety. In some cases, severe brown rugose symptoms are present on almost all fruits, and in other instances, symptoms are mainly found on the vegetative parts in the form of severe or mild mosaic, necrosis, leaf distortion, or other symptoms. The severity of the viral symptoms on tomato fruits has a high impact on tomato growers, since this new viral disease results in fruits of very poor quality and value, which are almost unmarketable. The ToBRFV is easily transmitted by mechanical means, which facilitates its rapid spread, and makes it difficult to control. Transmission of the ToBRFV is also likely to occur through infected seeds.

Patent application WO2019110130A1 to Rijk Zwaan discloses a Solanum lycopersicum plant that is resistant to ToBRFV, said plant comprises a QTL on chromosome 11, and/or a QTL on chromosome 12, and/or a QTL on chromosome 6.
Patent application US20200048655A1 to SEMINIS VEGETABLE SEEDS discloses Tomato plants exhibiting resistance to Stemphylium. The invention also provides a Solanum lycopersicum plant comprising a recombinant chromosomal segment on chromosome 11, wherein said chromosomal segment comprises a Stemphylium resistance allele from Solanum pimpinellifolium conferring increased resistance to Stemphylium to said plant compared to a plant not comprising said allele, and wherein said recombinant chromosomal segment further comprises a Tomato Brown Rugose Fruit Virus (ToBRFV) resistance allele. In some embodiments, said ToBRFV resistance allele is located within a chromosomal segment flanked by marker locus M1 and marker locus M3 on chromosome 11 in said plant. In other embodiments, said plant is homozygous for said ToBRFV resistance allele.
Patent application US20200077614A1 to VILMORIN & CIE discloses Solanum lycopersicum plant resistant to Tomato Brown Rugose Fruit virus comprising in its genome the combination of the Tm-1 resistance gene on chromosome 2, and at least one quantitative trait locus (QTL) chosen from QTL3 on chromosome 11, QTL1 on chromosome 6 and QTL2 on chromosome 9, that independently confer to the plant foliar and/or fruit tolerance to ToBRFV.
Patent application WO2020148021 to Enza Zaden Beheer B. V. discloses a plant of the S. lycopersicum species that is resistant to Tobamovirus, wherein the plant comprises one or more genomic sequences or locus providing said resistance.

In view of the prior art documents and given the various challenges faced by tomato growers around the world, there is still an unmet long-felt need to obtain tomato varieties which are resistant in a dominant manner to tomato brown rugose fruit virus.

### BRIEF DESCRIPTION OF THE INVENTION

To overcome the challenge of ToBRFV infection and related poor quality and value of fruits, attempts are made for discovering and/developing ToBRFV-resistant varieties.

In the present invention, novel genetic markers are presented conferring partially dominant resistance to ToBRFV.

The accompanying drawings, which are included to provide a further understanding of the invention and are incorporated in and constitute a part of this specification, illustrate embodiments of the invention and together with the description serve to explain the principles of the invention.

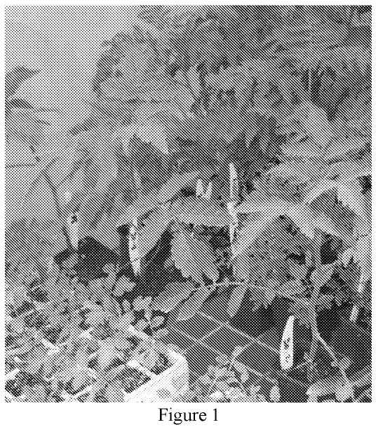

FIG. 1 depicting symptomless tomato plants exposed to ToBRFV, which are served to search resistant varieties according to the present invention;

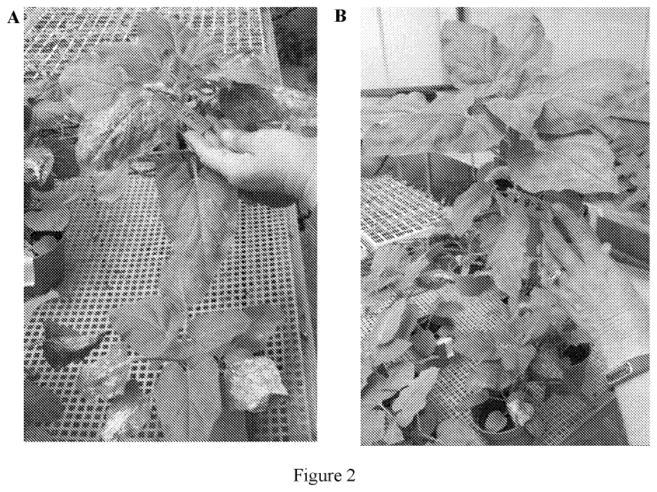

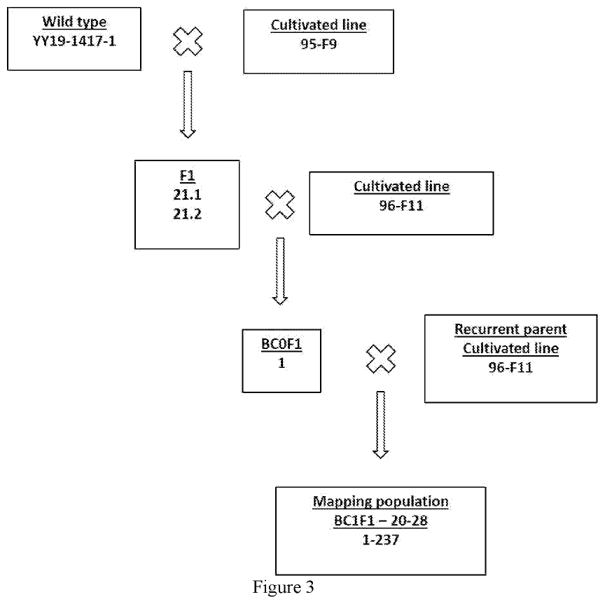

FIG. 2 depicting necrosis reaction on N. glutinosa leaves from symptomless tomato plants exposed to ToBRFV.

FIG. 3 depicting a flowchart detailing a crossing program used for generating the resistant plants of the present application;

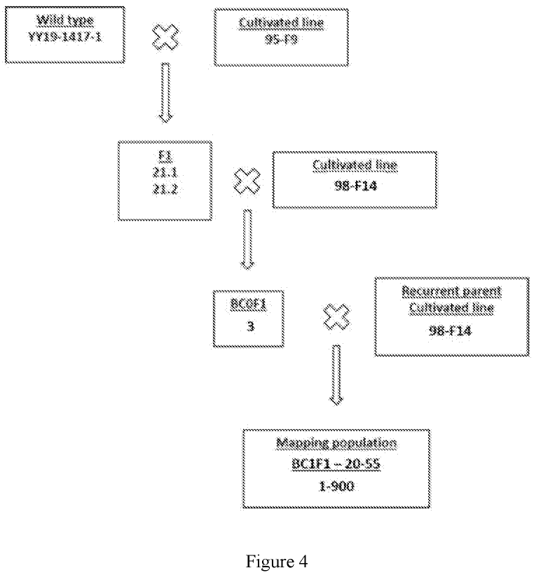

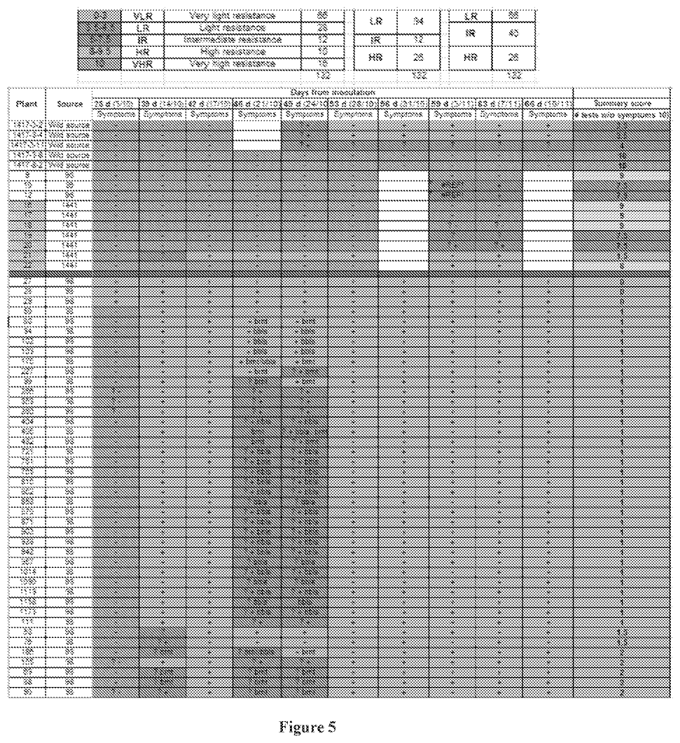

FIG. 4. depicting a flowchart detailing a crossing program used for generating the resistant plants of the present application;

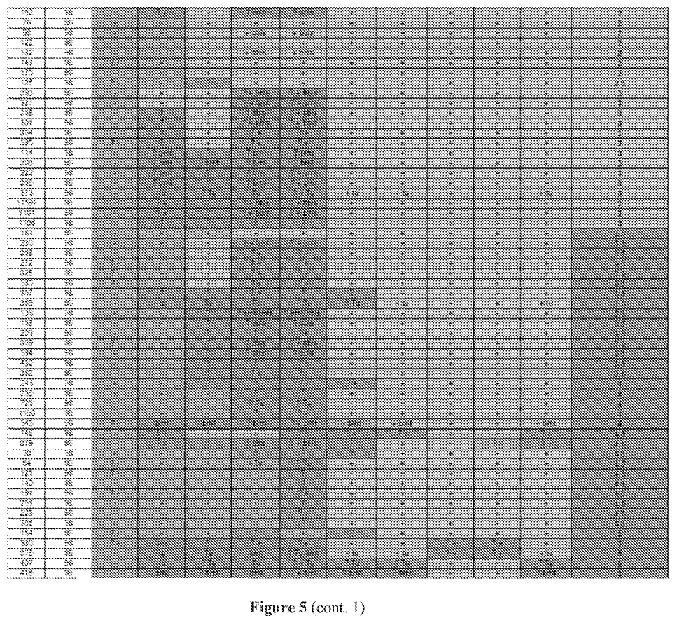

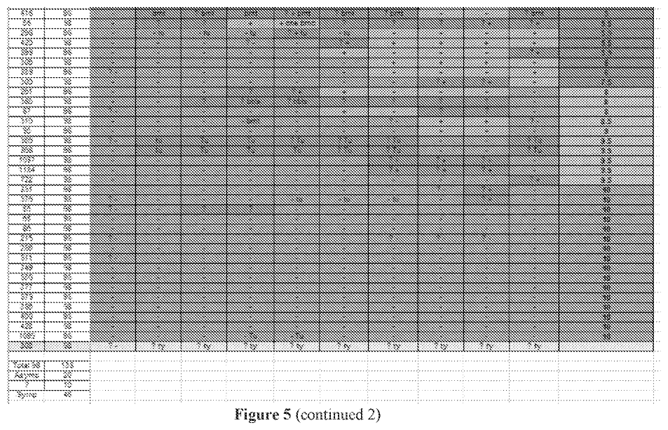

FIG. 5. depicting a phenotyping results collected on population BC1F1 98/20-55

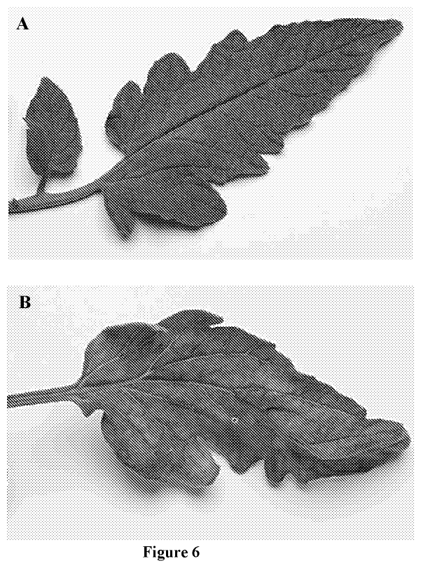

FIG. 6. depicting the ToBRFV-resistant tomato leaf of the present invention (FIG. 6A) compared to a ToBRFV-sensitive tomato leaf (FIG. 6B);

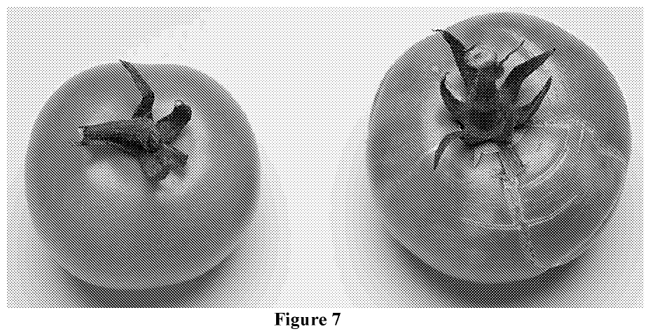

FIG. 7 depicting the ToBRFV-resistant tomato fruit of the present invention (left) compared to a ToBRFV-sensitive tomato fruit (right); and

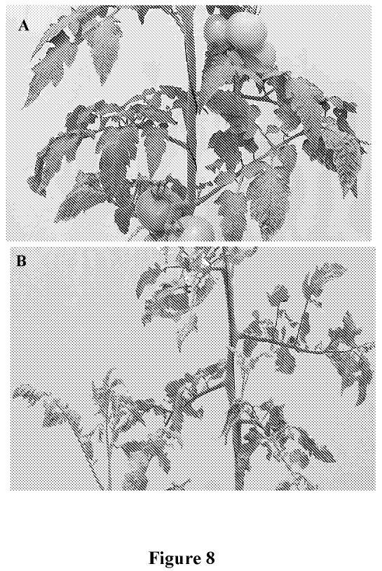

FIG. 8 depicting the ToBRFV-resistant tomato plant of the present invention (FIG. 7A) compared to a ToBRFV-sensitive tomato plant (FIG. 7B).

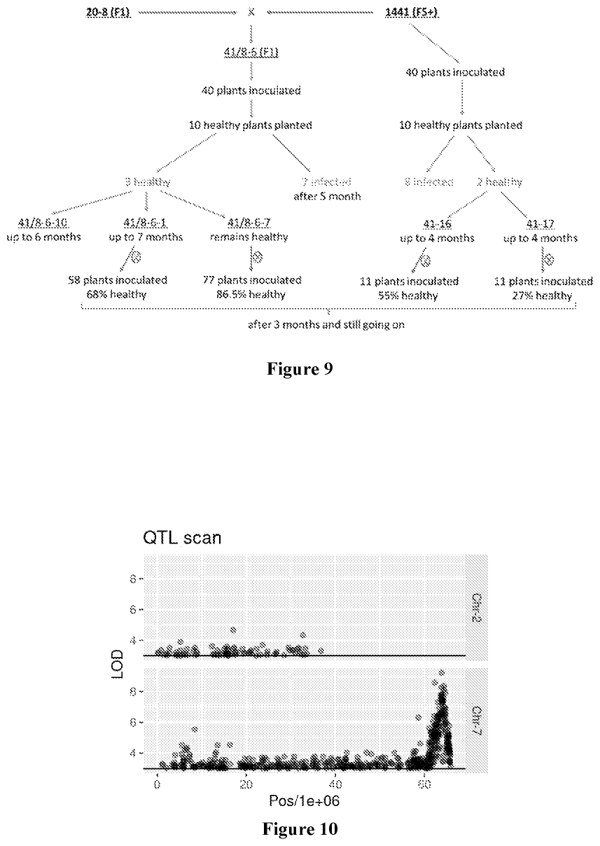

FIG. 9 provides a scheme of crosses that were conducted for ToBRFV resistance level evaluation of the addition resistance source line 1441

FIG. 10. depicting two ToBRFV resistance QTLs mapped on Chromosome 2 and chromosome 7 deriving from population BC1F1 20-55 illustrated via a Manhattan plot

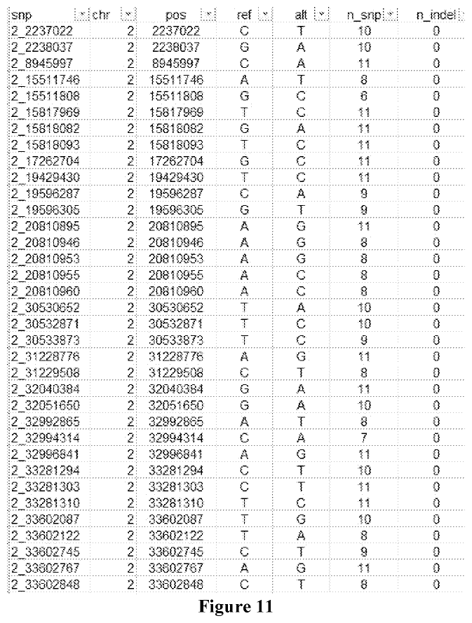

FIG. 11. depicting a list of SNP markers QTL mapped on Chromosome 2 and its positions

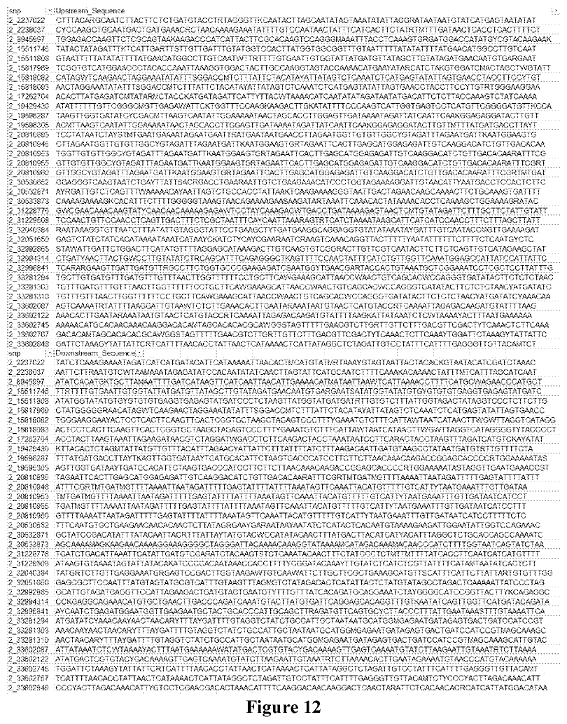

FIG. 12. depicting a list of SNP markers QTL mapped on Chromosome 2 and its positions

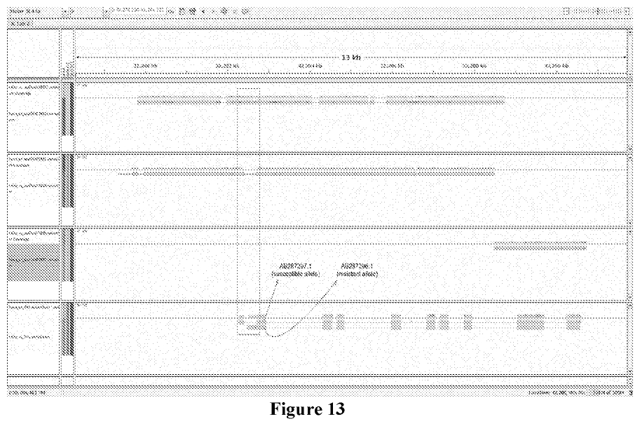

FIG. 13. depicting scaffolds mapping and alignment using IGV of TM1 allele mining deriving from 1441

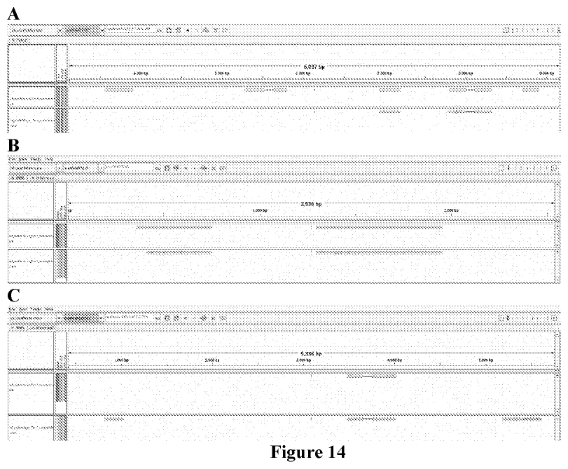

FIG. 14. depicting scaffolds comparisons and alignment to the known TM1 resistant and susceptible alleles: 14A. Comparison of scaffold73333 to resistant and susceptible alleles of TM-1. 14B. Comparison of scaffold77235 to the resistant and susceptible alleles of TM-1. 14C. Comparison of scaffold139913 to the resistant and susceptible alleles of TM-1

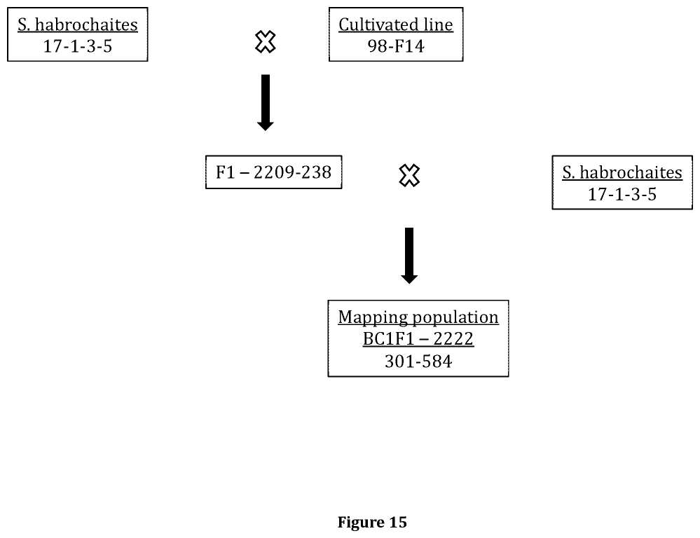

FIG. 15. depicting a flowchart detailing a crossing program used for generating the resistant plants of the present application

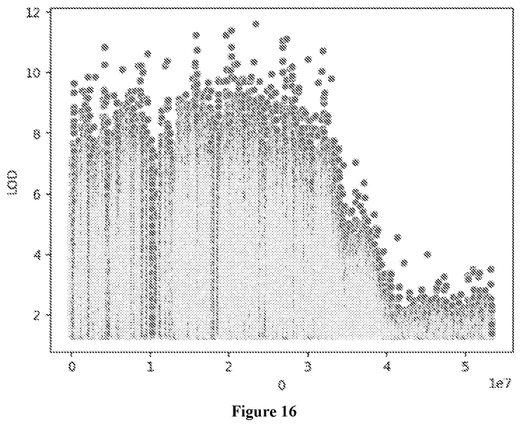

FIG. 16. depicting ToBRFV resistance QTL mapped on Chromosome 2 and chromosome 7 deriving from population BC1F1 2222 illustrated via a Manhattan plot

### SUMMARY OF THE INVENTION

It is one object of the present invention to disclose a tomato plant harboring a partially dominant resistance gene/genes to tomato brown rugose fruit virus (ToBRFV).

It is another object of the present invention to disclose the tomato plant as described above, wherein said plant is the offspring of crossing a ToBRFV-resistant wild tomato plant with a ToBRFV-susceptible cultivated tomato plant, or a ToBRFV-resistant wild tomato plant with a ToBRFV-susceptible wild tomato plant.

It is another object of the present invention to disclose the tomato plant as described above, wherein said ToBRFV-resistant wild tomato plant harbors said partially dominant resistance gene/genes.

It is another object of the present invention to disclose the tomato plant as described above, wherein said ToBRFV-resistant wild tomato plant is selected from a group consisting of LA0107 (S. corneliomulleri), LA0361, LA1918, LA2650 (S. habrochaites), LA1938, LA1969, LA2748, LA2755 and LA2931 (S. chilense).

It is another object of the present invention to disclose the tomato plant as described above, wherein said ToBRFV-susceptible cultivated tomato plant is Solanum Lycopersicum.

It is another object of the present invention to disclose the tomato plant as described above, wherein said plant is the offspring of crossing a ToBRFV-resistant cultivated tomato plant with a hybrid ToBRFV-resistant cultivated tomato plant.

It is another object of the present invention to disclose the tomato plant as described above, wherein said ToBRFV-resistant cultivated tomato plant is segregated from Solanum Lycopersicum cultivated line source 1441.

It is another object of the present invention to disclose the tomato plant as described above, wherein said hybrid ToBRFV-resistant cultivated tomato plant is the offspring of cultivated line crossed with a wild ToBRFV-resistant plant.

It is another object of the present invention to disclose the tomato plant as described above, wherein the crossing of cultivated source 1441 with a hybrid plant gives rise to a long-term resistance to ToBRFV for over 4 months.

It is another object of the present invention to disclose genetic markers for the use in identification and/or selection of tomato plants harboring partially dominant resistance to tomato brown rugose fruit virus (ToBRFV).

### DETAILED DESCRIPTION OF THE INVENTION

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a tomato plant which is resistant to tomato brown rugose fruit virus (ToBRFV) in a partially dominant manner.

The present invention relates to a tomato plant, wherein said ToBRFV resistance gene and/or one or more genomic sequences are as found in the deposit accession number NCIMB 43817. NCIMB 43817. L. esculentum x L. habrochaites Lycopersicum seeds were deposited on 16 Jul. 2021 at NCIMB Ltd, Ferguson Building, Craibstone Estate Bucksburn, AB21 9YA Aberdeen, United Kingdom.

As used herein after, the term “about” refers to any value being up to 25% lower or greater than the defined measure.

As used herein after, the term “tobamovirus” refers to a genus of plant viruses (comprising 35 species) belonging to the Virgaviridae family. These viruses are characterized by having a 300 nm long rod-shaped particle encapsulating a single-stranded, positive sense RNA genome of 6.2 to 6.4 kb encoding four proteins: the genomic segment expresses two replication-related proteins of 126 and 183 kDa, resulting from partial suppression of a stop codon; a 30-kDa movement protein (MP) is expressed through a sub-genomic RNA1 (sgRNA1); and a 17.5-kDa coat protein (CP) is expressed from a second sub-genomic RNA2 (sgRNA2). The tobamoviruses infect mostly plants belonging to the Brassicaceae, Cucurbitaceae, Solanaceae and Malvaceae families. The most known tobamoviruses which infect tomato plants are: the tobacco mosaic virus (TMV), tomato mosaic virus (ToMV) and tomato mild mottle virus (ToMMV), tobacco mild green mosaic virus (TMGMV) and pepper mild mottle virus (PMMoV).

As used herein after, the term “Tomato brown rugose fruit virus (ToBRFV)” refers to a recently discovered virus, belonging to the tobamoviruses genus. This virus was first discovered in the spring of 2015 in Jordan, and it was also found in Israel, Turkey, the Netherlands, Mexico, and USA. The genome of the virus comprises 6393-nt single-stranded RNA (ssRNA) encoding four proteins. The ToBRFV causes the following symptoms: severe brown rugose symptoms on fruits, yellow spots on fruits, severe or mild mosaic on leaves, leaf narrowing, necrosis, leaf distortion and more. The ToBRFV is easily transmitted by mechanical means, and through infected seeds.

As used herein after, the term “variety” or “varieties” refers to the usual denomination in agricultural industry and correspond to a plant of a given botanical taxon which is distinct from other existing plant, which is uniform and stable.

According to the international seed federation, “susceptibility” is the inability of a plant variety to restrict the growth and/or development of a specified pest. “Resistance” is the ability of a plant variety to restrict the growth and/or development of a specified pest and/or the damage it causes when compared to susceptible plant varieties under similar environmental conditions and pest pressure. Resistant varieties may exhibit some disease symptoms or damage under heavy pest pressure. Two levels of resistance are defined. “High resistance (HR)”: plant varieties that highly restrict the growth and/or development of the specified pest and/or the damage it causes under normal pest pressure when compared to susceptible varieties. These plant varieties may, however, exhibit some symptoms or damage under heavy pest pressure. “Intermediate resistance (IR)”: plant varieties that restrict the growth and/or development of the specified pest and/or the damage it causes but may exhibit a greater range of symptoms or damage compared to high resistant varieties. Intermediate resistant plant varieties will still show less severe symptoms or damage than susceptible plant varieties when grown under similar environmental conditions and/or pest pressure.

As used herein after, the term “partially dominant resistance” refers to inheritance of a dominant gene or genes from one parent, which confers more than partial resistance in the offspring to an infection.

As used herein, the term “homozygous” refers to a genetic condition or configuration existing when two identical or like alleles reside at a specific locus, but are positioned individually on corresponding pairs of homologous chromosomes in the cell of a diploid organism.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

The term “selfing” used herein refers in some embodiments to the production of seed by self-fertilization or self-pollination; i.e., pollen and ovule are from the same plant/parental line.

The term “phenotype” is understood within the scope of the invention to refer to a distinguishable characteristic(s) of a genetically controlled trait.

As used herein, the phrase “phenotypic trait” refers to the appearance or other detectable characteristic of an individual, resulting from the interaction of its genome, proteome and/or metabolome with the environment.

As used herein after, the term “Nicotiana glutinosa assay” refers to an assay designed for the detection of a plant virus. The assay comprises the inoculation of the indicator plant Nicotiana glutinosa leaves with extracts (such as sap) from infected plants. If the infected plants are resistant to the virus, the N. glutinosa leaves are not expected to develop any reaction to the virus. If the infected plants are sensitive to the virus, their extracts contain significant amounts of viral particles, and in contact with the N. glutinosa leaves, the leaves will necrotize.

As used herein after, the term “quantitative trait locus (QTL)” refers to a part of the DNA which correlates with a variation of a quantitative trait in the phenotype of a population of organisms. QTLs are mapped by identifying which molecular markers correlate with an observed trait.

The present invention provides tomato plants which exhibit resistance to the tomato brown rugose fruit virus (ToBRFV) in a partially dominant manner.

Plants can be targeted and infected by a variety of pathogens (bacteria, viruses, fungi etc.), and as a result, defense mechanisms conferring resistance have evolved. In general, those mechanisms comprise genes that sense the presence of a specific pathogen by recognizing specific components of that pathogen, referred to as “avirulence factors”. Triggering of the resistance genes can lead to defense pathways, such as the hypersensitive response, where the infected areas undergo programmed cell death and necrotic lesions aiming to eliminate the pathogen are observed. In some cases, the resistance mechanism is so intensive, that no lesions are apparent.

The resistance can be dominant or recessive (where all resistance gene copies are in dominant or recessive state, respectively.) In order for the progeny to inherit dominance resistance, it suffices that only one of the parental plants carries the dominant resistance gene/genes. Using a plant with dominant resistance to cross a susceptible plant facilitates the insertion of the dominance genes to elite hybrids.

It is also noteworthy that wild species used for production of resistant offspring by crosses often pass down undesired or disadvantageous traits which might manifest in the homozygote state as recessive traits. The present invention discloses ToBRFV-resistant tomato plants, wherein the resistance is dominant. This dominant resistance is inherited solely from one of the parental lines used to generate to tomato plants of the present invention, since one of the parental lines is a wild tomato species possessing said dominant resistance.

In a preferred embodiment of the present invention, the ToBRFV resistant tomato plants are produced by crossing a resistant wild tomato parent harboring a dominant resistance gene or genes with a susceptible cultivated tomato plant (Solanum Lycopersicum).

In yet another preferred embodiment of the present invention, the ToBRFV resistant parent used for said cross is the male parent, whose pollen grains are utilized to fertilize the cultivated tomato female plant.

In yet another preferred embodiment of the present invention, the ToBRFV resistant parent used for said cross is the female parent, whose ovules are fertilized with pollen of the male parent. In yet another preferred embodiment of the present invention, the progeny of said cross is symptomless when exposed to ToBRFV.

In yet another preferred embodiment of the present invention, the resistance to ToBRFV is passed down through the offspring of said cross via dominant resistance gene/genes.

In yet another preferred embodiment of the present invention, the dominant resistance to ToBRFV in cultivated lines of S. lycopersicum, could use to create ToBRFV resistant hybrid varieties.

In an additional preferred embodiment of the present invention, a ToBRFV resistant tomato plant is obtained by crossing a ToBRFV resistant wild tomato plant with a susceptible wild tomato plant.

In an additional preferred embodiment of the present invention, a ToBRFV resistant tomato plant is obtained by crossing a ToBRFV resistant hybrid with a resistant wild tomato plant.

In an additional preferred embodiment of the present invention, a ToBRFV resistant tomato plant is obtained by crossing a ToBRFV resistant hybrid with a ToBRFV resistant cultivated tomato plant.

In an additional preferred embodiment of the present invention, novel genetic markers are disclosed for the use in identification or selection of tomato plants harboring dominant resistance to tomato brown rugose fruit virus (ToBRFV).

In an additional preferred embodiment of the present invention, the novel genetic markers are configured to be linked to the gene or genes or QTL conferring dominant resistance to ToBRFV in said tomato plants.

### Example 1

An extensive screening of about 50 wild tomato species accessions, obtained from the tomato genetics resource center (TGRC) Gene bank at UC Davis, resulted in the identification of 9 accessions, exhibiting some forms of resistance to ToBRFV. The determination of resistance was carried out using the bioassay method which comprises: (i) lack of phenotypic symptoms after actively infecting the plants with the virus and (ii) no symptoms on Nicotiana glutinosa plants that were infected with the plant's sap.

Reference is now made to FIG. 1 depicting tomato plants which did not manifest any symptoms after being actively infected with ToBRFV. These symptomless plants are further examined in the Nicotiana glutinosa assay, during which the sap of these plants comes in contact with leaves of Nicotiana glutinosa to monitor existence of necrosis in response to the viral infection.

Reference is also made to FIG. 2 depicting Nicotiana glutinosa leaves which exhibit necrosis (FIG. 2A), and leaves which do not exhibit any signs of necrosis (FIG. 2B) after being exposed to sap from ToBRFV-infected symptomless tomato plants.

### Example 2

Plants from each of the inoculated accessions which exhibited resistance to the ToBRFV and successfully passed the bioassays described in example 1 of the detailed description, were grown in a greenhouse to multiply and were used as pollen source for crosses after retesting for absence of virus. Plants that manifested late symptoms or had any positive response in the N. glutinosa assay were eliminated. The resistant plants were derived from the following 9 accessions: LA0107 (S. corneliomulleri), LA0361, LA1918, LA2650 (S. habrochaites), LA1938, LA1969, LA2748, LA2755 and LA2931 (S. chilense).

A research program was set up to determine if the resistance could be transferred to Solanum Lycopersicum, and to identify the genetics underlying the ToBRFV resistance within the different accessions. One obstacle to overcome is that not all wild species cross easily with S. Lycopersicum to set fruit that contain fertile seeds (even if seeds are created, they might not germinate because of inherent incompatibility).

### Example 3

As mentioned above, to investigate the genetic basis of Tomato brown rugose fruit virus (ToBRFV), the inventors screened about 50 wild tomato sources including S. arcanum, S. chilense, S. corneliomulleri, S. habrochaites, S. huaylasense, S. pennellii and S. peruvianum.

Mapping populations design and phenotype characterization The following crossing programs are presented in FIG. 3 and elaborated in the following disclosure: crossing between the line YY19-1417-1 (S. habrochaites) with one of Philoseed parental line 95-F9 show high resistance (HR) to ToBRFV. This phenotype indicates a dominant mode of inheritance.

The F1 plants were crossed with different esculentum backgrounds representing Philoseed parental lines to produce BC0F1 and BC1F1 seeds. The BC0F1 plants show segregating phenotype, as well as, on generations BC1F1, BC1F2 and BC2F1.

The inventors used a mapping population 98/20-55 (FIG. 4) which includes 880 BC2F1. The population (98/20-55) was generated by crossing the F1 plants with the parental line 98-F14 (indeterminate cluster type tomato) generating BC1F1 and then backcrossing a single (HR) BC1F1 plant with its recurrent line 98-F14 to produce the BC2F1 mapping population. It is important to mention here that population 20-55 is genetically BC2F1 and not BC1F1 since the initial cross was done on line 95-F9 and the Backcross was done on different line (98-F14), therefore, the ratio of the wild background in the mapping populations is accordingly, about 12.5%.

### Tissue Sampling for Mapping the Trait

Leaf samples from single plants of the 4 parents of the mapping population (two of each, BC0F1-1+98-F14, and BC1F1-8+95-F9, respectively) were sent for DNA extraction and whole genome sequencing by NRGene Ltd. The leaf samples of all the plants from the two mapping populations were sent for DNA extraction and genotyping. The phenotypes of the mapping populations were sent to NRGene Ltd. for the mapping analyses and is presented in FIG. 5.

### ToBRFV Resistance Phenotype Characterization

The inoculum used is the one isolated and maintained by Dr. Aviv Dombrovsky from the Agricultural Research Organization, Volcani Center, Israel. An inoculation of the seedlings conducted at 1-2 real leaves stage and the evaluation of resistance was done after 3-4 weeks as described at Avner Zinger et. al., Plants 2021, 10, 179., 2021. The determination of resistance was carried out using the bioassay method which comprises: (i) lack of phenotypic symptoms after actively infecting the plants with the virus and (ii) no symptoms on Nicotiana Glutinosa plants that were infected with the infected plant's sap.

FIG. 6-8 depict the ToBRFV-resistant tomato plant of the present invention (generated according to the crossing programs above) compared to a sensitive tomato plant.

### Example 4

To proceed with obtaining a ToBRFV-resistant cultivated tomato plant, wherein the resistance involves a dominant gene or genes, the following steps were taken:
a. Generating F2 and F3 populations of the susceptible cultivated line x S. Habrochaites accession, and backcross populations, for QTL/gene mapping of this dominant resistant cross. The bioassays described in example 1 (monitoring symptoms after an infection and the Nicotiana glutinosa assay) are executed on all generations to confirm the presence of dominant resistance. Once the QTL or gene/genes conferring dominant resistance to ToBRFV are identified, they could be utilized as novel genetic markers. Those markers could be harnessed to rapidly identify and select ToBRFV-resistant tomato varieties/cultivars without the need to perform the above-mentioned bioassays, which are time-consuming and laborious.
b. In addition to testing resistant populations derived from crossing the susceptible cultivated line x S. Habrochaites accession, a further goal of this invention is to germinate F1 hybrids of susceptible cultivated lines crossed with the other 8 resistant wild species accessions, which are mentioned in example 2. The F1 hybrids are infected with ToBRFV and determined to see if they harbor resistance dominance, by monitoring symptoms and conducting genetic analyses such as QTL mapping to discover the dominant resistance gene or genes.
c. Another step of the present invention is producing germinating F1 hybrids of susceptible wild accessions lines crossed with resistant plants derived from the 9 accessions (LA0107, LA0361, LA1918, LA2650, LA1938, LA1969, LA2748, LA2755 and LA2931). After generating these crosses, the hybrid F1 plants are infected with ToBRFV and determined to see if they harbor resistance dominance. This mediating cross is designed to facilitate obtaining fertile F1 seeds that then could be crossed with S. lycopersicum cultivated lines.
d. An additional step of the present invention is crossing the resistant plants of the above 9 accessions with the F1 hybrid (disclosed in example 2) that already germinated and verified for being resistant. These types of crosses are destined to achieve two goals: (i) a bridging cross to overcome germination problems by creating a germinating 3-way cross; and (ii) investigating the resistance obtained by combinations of different resistance sources.

### Example 5

In parallel to an assortment of wild species that were tested for resistance to ToBRFV a large range of cultivated breeding material was tested. Within the cultivated material two plants that segregated from S. lycopersicum cultivated line source 1441 (details bellow) were found to show the best resistance by staying negative on N. glutinosa (bioassay) for over 4 months. These 2 plants (designated 41-16, 41-17) were combined into the breeding program for ToBRFV resistance together with the wild accession that was found, that showed a high level of resistance compared to all the others that we tested. FIG. 9 provides a scheme of crosses that were conducted for ToBRFV resistance level evaluation of the addition resistance source line 1441.

As described on FIG. 9, 40 young seedlings of source 1441 were inoculated two weeks from sowing. After 3 weeks all were healthy by the bioassay test. 10 of 40 seedlings were planted in an infected greenhouse and were followed along the growing season.

8 plants of the 10 became infected during 2 months from inoculation and 2 plants remained healthy for about 4 months: 41-16, 41-17. These 2 resistant plants were integrated into the breeding program. A new cross named 41/8-6 was created, which is a cross of 1441 with F1 plant 20-8 (hybrid of the applicant which is a cross of cultivated line with the resistant wild accession). 40 of the F1 seedlings were inoculated two weeks from sowing. After 3 weeks all were healthy by the bioassay test.

10 of 41/8-6 plants were planted in an infected greenhouses and followed along the growing season. All the 10 plants remained healthy for over 4 months. After 5 months, 3 remained healthy, designated as 41/8-6-1, 41/8-6-7, 41/8-6-10. The last one became infected after 6 months, plant 41/8-6-1 became infected after 7 months and plant 41/8-6-7 remained healthy until the end of growing season.

58 and 77 seedlings of line 41/8-6-1 and 41/8-6-7, respectively, were created and inoculated two weeks from sowing. After 3 weeks all were healthy by the bioassay test and planted in an infected greenhouse. After 3 months from infection, 68% and 87% of the plants from line 41/8-6-1 and 41/8-6-7, respectively were healthy by the bioassay test.

### Tobamovirus Resistance QTL Analysis

### Tissue Collection, DNA Extraction and Sequencing

### Population 4, BC1F1—98/20-55

A total of 10 plates containing 880 tissue samples of the resistant trait segregating population were sent to Gene-G for DNA extraction.

The quality and quantity of 837 samples of 880 DNA extraction were sufficient for Skim-Sequencing library preparation and sequencing requirements. DNA samples were sent to Psomagen, Inc. (1330 Piccard Drive, Ste 103, Rockville, MD 20850) in the US for sequencing.

Parental line triplicate DNA samples was pooled and QCed, Next TruSeq DNA PCR Free sequencing libraries were prepared. Libraries were sequenced on an Illumina NovaSeq 6000 sequencer with 150 bp paired end reads, to ˜x35 coverage calculated by a genome size of 0.9 Gbp.

Progeny DNA samples were QCed and iGenomX Riptide sequencing libraries were prepared. Libraries were sequenced on an Illumina NovaSeq 6000 sequencer with 150 bp paired end reads, to ˜x2 coverage calculated by a genome size of 0.9 Gbp.

Sequencing data was uploaded to applicant's AWS-S3 cloud storage. Sequencing data coverage cross samples qualified applicant's requirements for QTL analysis.

The FastQC software has been run on the sequencing data to estimate the quality of the raw data. The quality indicators of the data have met with applicant's standards requirements.

### Phenotype of Population 4 (BC1F1—98/20-55)

Phenotype data of the mapping populations is presented in Table 1 below:

TABLE 1

| Phenotype | R score | Count |
|---|---|---|
| S | 0 | 707 |
| R1 | 1 | 46 |
| R2 | 2 | 55 |
| R3 | 3 | 23 |

In order to have a balanced phenotype distribution in QTL search, the ToBRFV resistance scores were clustered into 4 categories as depicted in Table 1. The number of samples in each category is indicated in the Count column. The S category indicates the sensitive trait value and R1-R3 indicate the resistance trait values. R1 is the least resistant while R3 is the most resistant. R-score from the table was used as the phenotype value in the search for QTL.

### Resistance Categories:

S=Susceptible plants=R Score 0. The plants in that group were displaying ToBRFV symptoms 28 days after inoculation. Only 40 plants were transplanted in the field.
R1=Very light resistance=summary score 1-2. The plants in this group were asymptomatic up to 42 days after inoculation.
R2=Intermediate Resistant plants=summary score 3-7 None/late age asymptomatic and up to 56 days after inoculation.
R3=High Resistant plants=summary score 8-10. The plants in this group were asymptomatic up to 66 days after inoculation.

The phenotype data is specified in FIG. 5.

### Computational Analysis:

### Preliminary Definitions

1) As used herein after, the term “K-mer” refers to a substrings of length k contained within a biological sequence. In bioinformatics, k-mers are composed of nucleotides and primarily used as a tool in computational genomics, sequence analysis, and DNA assembly. The frequency of a set of k-mers in a species' genome, or in a genomic region, or in a class of sequences can be used as a “finger print” of the underlying sequence. Comparing these frequencies is computationally advantageous compared to sequence alignment and is an important method in alignment-free sequence analysis.
2) As used herein after, the term “Basic Local Alignment Search Tool (BLAST)” refers to a computational search program configured to find regions of local similarity between sequences. The BLAST program compares nucleotide or protein sequences to sequence databases and calculates the statistical significance of matches. BLAST can be used to infer functional and evolutionary relationships between sequences as well as help identify members of gene families (blast.ncbi.nlm.nih.gov/Blast.cgi).

As used herein after, the term “logarithm of the odds score (LOD score)” refers to a statistical numerical estimate of whether two genetic loci are (two genes, or a gene and a disease) physically close enough to each other on a particular chromosome that they are likely to be inherited together. A LOD score of 3 or higher is generally understood to mean that two genes are located close to each other on the chromosome and are most likely linked. In terms of significance, a LOD score of 3 means the odds are 1,000:1 that the two genes are linked and therefore inherited together.

As used herein after, the term “p-value” refers to a measure of the probability that an observed difference could have occurred just by random chance according to the null hypothesis significance testing. The lower the p-value, the greater the statistical significance of the observed difference. P-value can be used as an alternative to or in addition to pre-selected confidence levels for hypothesis testing.

### Data Pre-Processing

The genome of the susceptible parental line (98_F16-2) was assembled to a chromosome level using NRgene Assembly technology, using the Heinz genome assembly (version SL4.0) as reference. The reference genome used in the pipeline was downloaded from https://solgenomics.net/organism/Solanum_lycopersicum/genome (version SL4.0) Briefly, the genome was assembled to a contig level and the contigs were mapped to the reference genome based on sequence identity.

Since the sequencing data of resistant donor line was not available, the assembly we generated an assembly representation containing both parental lines genome sequences. We accomplished this by concatenating the Skim sequencing data of 200 progeny lines (100 resistant and 100 susceptible) and used this combined sequencing data set as input for our assembly pipeline. Out of this assembly representation we extracted the sequences of the resistant donor line by filtering out sequences that were found in the susceptible parental line assembly (98_F16-2).

The assembled contigs were split into non-overlapping K-mers of 74 bp and a K-mer database (KDB) for each parental line was constructed holding all the corresponding K-mers.

The KDB for each line was filtered by comparing the K-mers between the different lines and keeping unique K-mers that appear exactly once in the corresponding line and none in the other lines.

This was done using an exact match comparison between any two K-mers. Each unique K-mer is then searched in the progeny to generate an absence/presence table for each K-mer and Progeny-sample.

Next, K-mer counts were aggregated the by contigs. A Contig-level Count Matrix (CCM) was constructed with presence/absence values per sample in the progeny. The values in CCM were normalized by sample sequencing depth and the number of contig K-mers to generate Contig-level Genotyping Matrix (CGM) having presence/absence values for each sample on each contig.

This pipeline produces a CGM that represents contigs originating from the wild tomato accession genome since all other sequences from the cultivated lines were filtered out.

### QTL Analysis

QTL analysis was performed using a regression model on each of the contigs using the values stored in the CGM. This regression is related to the wild sample inheritance patterns as CGM represents the alleles coming from the wild sample only. R-score was used from Table 1 as the phenotype value in the search for QTL.

A QTL scan was performed by regressing the phenotype score on the genotype at each contig. A significant QTL was declared if a model including the genotype was substantially better than a model without the genotype using a likelihood-ratio test.

A LOD score was calculated by comparing the variance explained by the two models as indicated in Broman et al. (Ch 4.1, Pg77). A Threshold of LOD>3 was used to declare significant results. Contigs that passed the LOD>3 threshold were subject to further examination and confidence intervals were calculated for the selected peaks based on 1.5 units in LOD scores from the most significant contig.

LOD scores at each contig are presented in FIG. 10. The panels describe the different chromosomes based on the reference. Only contigs that were successfully mapped to the reference genome are shown. A horizontal line indicating LOD=3 is indicated in black.

Sequences from the wild accession are present in a significant proportion on chromosomes 7, and 2. On chromosome 7 a significant peak with LOD>8 was found. At the peak the explained phenotypic variance is 5%. There is evidence in the LOD score curve that the source of this correlation is at the end of the Chromosome. The 95% confidence interval for the suggested region is at: Chr7:62-65 [Mbp]. An additional QTL was found on Chromosome 2 with LOD=4. The variance explained by both QTLs is 7.4%. The QTL on chromosome 2 spans the first arm with a confidence interval of Chr2:0-43 [Mbp].

### Genotype/Phenotype Counts

Table 2 below discloses the effect of each genotype on the phenotype at the peak of the QTL loci. The Genotype columns indicate the presence/absence of a selected QTL: 0 indicates absence and 1 indicates presence. The Phenotype Score (0-3) holds the number of samples found in each category.

The table suggests that the major effect is created by the QTL on chr-7 and the addition of chr-2 provides an additional protection.

TABLE 2

| | |
|---|---|
| Genotype | Phenotype Score |

| qtl-chr7 | qtl-chr2 | 0 | 1 | 2 | 3 |
|---|---|---|---|---|---|
| 0 | 0 | 168 | 6 | 1 | 0 |
| 0 | 1 | 174 | 8 | 6 | 2 |
| 1 | 0 | 184 | 11 | 13 | 6 |
| 1 | 1 | 181 | 21 | 35 | 15 |

### Marker Sequences

Unique sequences that could serve as molecular markers for the ToBRFV resistance were chosen based on the sequences of the contigs associated ToBRFV resistance. More specifically, a close inspection was carried out on short sequences that were present in the contigs and were not present in other samples that are not resistant. This was done by finding single nucleotide variants (SNVs) present in these contigs (when compared to a reference genome), which were not present in other tomato lines, and extracting the flanking sequences around these SNVs, as briefly described below.

First, contigs that were associated with ToBRFV resistance near the QTL regions (based on QTL analysis of the CGM; see above) were selected and variants (in respect to a SL4.0 reference genome) were extracted from these sequences. This was done by aligning these contigs to the reference genome and extracting all the differences between the reference genome and the aligned contigs. Alignment was done by both BWA (https://github.com/lh3/bwa) and minimap2 (https://github.com/lh3/minimap2) tools and onl markers that were detected by both methods were selected. A search was for single nucleotide variants (SNVs) and not INDELs, as they are less accurate for genotyping.

In order to confirm that these SNVs were unique to the resistant line and were not present in other non-resistant lines, assemblies of 38 other tomato lines that are not presumed to possess ToBRFV resistance were used. These lines include Tomato genome assemblies available to NRGene from proprietary and public databases containing both cultivated and wild accessions, some of which were tested and found susceptible to ToBRFV. Each of the assemblies sequences was divided into short contigs (of maximal length of 20,000 bps) and aligned the to the reference genome (SL4.0) using BWA and minimap2 tools. Similar to the procedure applied above to the contigs associated with ToBRFV resistance, variants were extracted based on these alignments. Each SNV that was detected in both the contigs associated with ToBRFV resistance and the contigs from the 38 “susceptible” tomato genomes assemblies was removed from the analysis as it was not unique to ToBRFV resistance.

Finally, the unique sequences that serve as marker sequences were determined based on these SNVs and additional 100 bps from each side of the SNVs. Search was only for SNVs with no INDELs and with up to 10 SNVs in their flanking regions. SNVs in the flanking regions were coded in standard IUPAC codes (Johnson, 2010; https://www.ncbi.nlm.hih.gom/pmc/articles/PMC2865858/). The presence and absence of SNVs/INDELs in the flanking regions was determined by the union of all variants detected in any of the 38 lines, as well as the contigs associated with ToBRFV resistance.

A list of these sequences with the different alleles in the SNVs in the middle of the sequences is provided FIG. 11 and FIG. 12, respectively. The list of markers driving from Chromosome 7 is specified in Table 3 below:

TABLE 3

| snp | chr | pos | ref | alt | n_snp | n_indel |
|---|---|---|---|---|---|---|
| 7_62366657 | 7 | 62366657 | C | A | 10 | 0 |
| 7_62366726 | 7 | 62366726 | G | T | 9 | 0 |
| 7_62366776 | 7 | 62366776 | T | C | 9 | 0 |
| 7_62366783 | 7 | 62366783 | T | C | 9 | 0 |
| 7_63089004 | 7 | 63089004 | A | G | 8 | 0 |
| 7_63693978 | 7 | 63693978 | T | A | 11 | 0 |
| 7_63699984 | 7 | 63699984 | C | G | 11 | 0 |
| 7_63700517 | 7 | 63700517 | T | C | 11 | 0 |
| 7_63700894 | 7 | 63700894 | A | G | 8 | 0 |
| 7_63831908 | 7 | 63831908 | G | A | 11 | 0 |
| 7_63832153 | 7 | 63832153 | A | G | 2 | 0 |
| 7_63836137 | 7 | 63836137 | A | C | 11 | 0 |
| 7_63862001 | 7 | 63862001 | A | T | 10 | 0 |
| 7_63863478 | 7 | 63863478 | C | T | 7 | 0 |
| 7_63863627 | 7 | 63863627 | C | T | 4 | 0 |
| 7_63864134 | 7 | 63864134 | G | A | 2 | 0 |
| 7_63864287 | 7 | 63864287 | T | C | 5 | 0 |
| 7_63864687 | 7 | 63864687 | A | G | 5 | 0 |
| 7_63864716 | 7 | 63864716 | A | C | 5 | 0 |
| 7_63864821 | 7 | 63864821 | C | G | 6 | 0 |
| 7_63864827 | 7 | 63864827 | A | G | 6 | 0 |
| 7_63864909 | 7 | 63864909 | A | C | 4 | 0 |
| 7_63896156 | 7 | 63896156 | T | A | 11 | 0 |
| 7_63914934 | 7 | 63914934 | C | A | 3 | 0 |
| 7_63940578 | 7 | 63940578 | A | G | 11 | 0 |
| 7_63942154 | 7 | 63942154 | A | G | 3 | 0 |
| 7_63942372 | 7 | 63942372 | G | A | 9 | 0 |
| 7_63991497 | 7 | 63991497 | A | G | 4 | 0 |
| 7_63991694 | 7 | 63991694 | C | T | 7 | 0 |
| 7_64126156 | 7 | 64126156 | G | T | 8 | 0 |
| 7_64126162 | 7 | 64126162 | A | G | 8 | 0 |
| 7_64128897 | 7 | 64128897 | G | A | 8 | 0 |
| 7_64163350 | 7 | 64163350 | C | A | 10 | 0 |
| 7_64163380 | 7 | 64163380 | C | T | 10 | 0 |
| 7_64167351 | 7 | 64167351 | C | T | 8 | 0 |
| 7_64241595 | 7 | 64241595 | T | C | 5 | 0 |
| 7_64241657 | 7 | 64241657 | T | G | 4 | 0 |
| 7_64243487 | 7 | 64243487 | T | C | 9 | 0 |
| 7_64361169 | 7 | 64361169 | A | T | 6 | 0 |
| 7_64361172 | 7 | 64361172 | C | T | 6 | 0 |

### Genetic Analysis of ToBRFV Resistance in Resistant Donors

Mapping of the QTLs controlling the resistance against ToBRFV in two resistance donor backgrounds was conducted in S. habrochaites line 17-1-3-5 and S. lycopersicum cultivated line 1441. The S. habrochaites lines used are not self-pollinated, so some level of heterozygosity was expected. The S. lycopersicum cultivated line 1441 is self-pollinated and was expected to be higher homozygosity level compared to the 17-1-3-5 source.

Two different research approaches were taken to reveal the genetic components in each donor background. For the S. habrochaites 17-1-3-5 background, a BC1F1 population was created, inoculated and phenotyped for disease symptoms. QTL analysis was performed using NGS of the whole population. For the 1441 background, WGS was conducted, followed by allele mining.

The QTL analysis made on the S. habrochaites 17-1-3-5 background via the BC1F1 2222 population revealed a segment on the chromosome 2 ranging from 0 to 35 MBp and 56 unique SNP markers were filtered and designed to describe its presence.

The allele mining of the 1441 source revealed a unique TM1 allele and described in eight markers on Chromosome 2.

The genetic components found with S. habrochaites 17-1-3-5 and 1441 on chromosome 2 are novel to the ToBRFV resistance efforts.

### 1441 Source Genetic Background

Tomato line 1441 exhibits a novel phenotype of high resistance to TOBRFV disease. This resistance differs from previously known mechanisms, such as the TM1 allele, suggesting the presence of a novel genetic factor. The successful identification and characterization of this new allele will provide a valuable resource for breeding programs seeking to develop TOBRFV-resistant tomato varieties.

Bioinformatics techniques to identify the genetic basis of the resistance phenotype in tomato line 1441, as described in the following steps:

### Genome Assembly

Genomic DNA from tomato line 1441 was sequenced to a depth of 30× coverage using 2×250 bp paired-end sequencing. These sequences were subsequently assembled into a scaffold-level assembly, enabling the mapping of known TM-1 gene sequences onto the 1441 genome. The scaffold assembly was also anchored onto the Heinz reference genome as part of the assembly process. This revealed that the scaffolds scaffold139913 and scaffold73333 of 1441 genome mapped to the same region on the Heinz reference genome (suggesting duplication in 1441), whereas the scaffold scaffold77235 of 1441 genome was found to be immediately adjacent to the other two scaffolds (FIG. 13). This suggests the presence of a duplication in 1441 that is not present in Heinz. One possible explanation for this phenomenon could be the insertion of a transposable element or other structural variant in 1441 that resulted in the duplication of scaffold77235. This duplication might be correlated with the high resistance phenotype. A visual inspection of the locus through IGV, identified that the scaffold139913 shows an allele that is not present in either its homologous scaffold (scaffold73333) or any of the susceptible and resistant TM1 alleles.

### Discovering the Region of Interest

1441 genome was investigated by aligning it to the known TM-1 genes for the susceptible and resistant alleles. This analysis revealed that the three scaffolds of 1441 genome assembly contain the TM-1 gene: scaffold139913, scaffold73333, and scaffold77235. Further, it was identified that the resistant allele (AB287296.1) is completely identical to scaffold73333 and scaffold77235 of 1441 genome (the two homologous scaffolds) with a few mutations in the susceptible allele (AB287297.1). Whereas mutations were found in both the resistant and susceptible allele in the alignment to scaffold139913. FIG. 14A Comparison of scaffold73333 to resistant and susceptible alleles of TM-1. This scaffold is shown to be completely identical to the resistant allele, with mutations present in the susceptible allele. FIG. 14B discloses comparison of scaffold77235 to the resistant and susceptible alleles of TM-1. This scaffold is shown to be completely identical to the resistant allele, with mutations present in the susceptible allele. FIG. 14C discloses comparison of scaffold139913 to the resistant and susceptible alleles of TM-1. This scaffold is shown to have mutations present in both the susceptible and resistant alleles.

Following the discovery of the scaffold139913, scaffold73333, and scaffold77235 in the 1441 genome, the next step was to determine: 1) whether the allele discovered in scaffold139913 was novel or had previously been identified in other tomato varieties, 2) whether the duplication that was found in 1441 (with scaffold139913 and scaffold73333 aligning to the same region) is unique to 1441. To do this, the three scaffolds were aligned to various known reference genomes, using NRGene's SplitChimera tool. Some of these reference genomes were taken from a tomato pan-genome consortium that include FG02_188, FLA7804, Heinz, LA0407, LA1416, LA3846, LA4354, NC_1_Grape, OH8245, and TS_1.

Through this mapping analysis, it was found that all three scaffolds from 1441 were positioned on chromosome 2 of the respective reference genomes, and their positioning was similar across the genomes. Detailed information about the positions is disclosed in Table 4 below:

TABLE 4

| | scaffold139913 (−) | scaffold73333 (+) | scaffold77235 (−) |
|---|---|---|---|
| Heinz | 32,288,576-32,281,344 | 32,280,192-32,288,000 | — |
| FG02_188 | 32,223,616-32,216,704 | 32215104-32223040 | 32,225,280-32,223,680 |
| FLA7804 | 33,146,496-33,139,584 | 33,137,984-33,145,920 | 33,148,160-33,146,560 |
| LA0407 | 33,659,840-33,599,808 | 33,623,552-33,659,584 | 33,661,760-33,659,904 |
| LA1416 | 35,000,704-34,994,048 | 34,992,960-35,000,896 | 35,003,200-35,001,664 |
| LA3846 | 33,466,880-33,458,432 | 33,458,368-33,466,304 | 33,468,544-33,466,944 |
| LA4354 | 33,686,208-33,678,400 | 33,678,336-33,686,272 | 33,688,576-33,687,040 |
| NC_1_Grape | 33,260,608-33,252,928 | 33,252,864-33,260,800 | 33,262,784-33,261,504 |
| OH8245 | 33,313,344-33,306,816 | 33,305,728-33,313,472 | 33,315,520-33,314,240 |
| TS_1 | 33,286,912-33,279,680 | 33,278,528-33,286,336 | 33,288,256-33,287,104 |

Further, it was observed that the duplication present in 1441 (with the two scaffolds scaffold139913 and scaffold73333) was unique to this variety, as no other varieties seemed to carry this duplication.

Given these results, scaffold139913 was chosen for marker design, as it appears to have a unique allele that is not present in scaffold73333 (FIG. 1). This evidence suggests that scaffold139913 may contain the genetic basis for the resistance phenotype observed in tomato line 1441, making it a promising candidate for further analysis.

### Marker Design Process

To design candidate markers for the novel resistance allele in tomato line 1441, the genomic variation of 1441 was compared to multiple known reference genomes using NRGene's contig-variant-calling pipeline. This pipeline generates variant call format (VCF) files for each genome. which contain information about the differences between that genome and a reference genome (in this case, Heinz SL4).

These VCF files were used to create a reference panel, which allowed to identify unique variations present in 1441 that were not present in the other reference genomes. A close investigation was carried oud on the region of the genome where the known TM1 gene anchors on 1441, as this location is likely to contain the novel allele responsible for the high resistance phenotype. The analysis revealed that there is a duplication in this region that is not present in other reference genomes. This duplication, along with the potential presence of the novel allele, makes this region a key area of interest in the search for the genetic basis of resistance in 1441.

To select candidate markers, SNPs that had no variants in the surrounding 50 base pair flanking region were selected, as these would be more specific for detecting the presence of the novel allele in 1441. In addition, SNPs that were heterozygous in 1441 and homozygous reference or no call in the other varieties were selected, as this helped to target markers that would specifically detect the presence of the novel allele due to the aforementioned duplication. Finally, a visual inspection was performed on the reference panel using the Integrative Genomics Viewer (IGV) to identify promising candidates for further consideration.

The novel Tm1 allele was found on scaffold 139913, while the wild type sequence was found on scaffold 73333. The list of markers and its details are found in Table 5 below:

TABLE 5

| | | | Contri- | |
|---|---|---|---|---|
| Marker | Genomic | | buting | |
| ID | position | Sequence | allele | |
| SNP_1 | chr2:32280274 | TGTTTGAACAATTTGTGCTCTCTA | T | scaffold139913 |
| | | GTTTCCAGGTAATTAGCAGCAAGA | | |
| | | AG[C/T]CTGAAGCCACTGGGAGT | | |
| | | ACAATATGACRAATGAATGTGYAT | | |
| | | GTCCATCCK | | |
| SNP_2 | chr2:32280745 | TAAMGCAAGGWAAAACAAACTAAA | A | scaffold139913 |
| | | TTCAAAGGGACTTACCTTGGTTTC | | |
| | | TT[G/A]TTGATTAGAACCATCTG | | |
| | | GAGGAGAATCATTTGTTCGAYTAG | | |
| | | CCAAATTAK | | |
| SNP_3 | chr2:32281634 | GAGAACCTGCTGAAGAGGTTTTTG | C | scaffold73333 |
| | | AGGCCAAAGAAGAGATAATCATAC | | |
| | | AA[T/C]TCTTGCGGCAGCAACTC | | |
| | | ATTTGCAACGGTCCGAACAACCAT | | |
| | | GGTTGTTGC | | |
| SNP_5 | chr2:32286031 | TACTTCGTTGGCCATCTCAAGTAC | G | scaffold73333 |
| | | AATGGCATTTGCATCAGCAAAGGG | | |
| | | CA[A/G]TAGACCAGCTAAGGATC | | |
| | | CCCTTCCTGCCATCCTAAAGCGCC | | |
| | | CTGAGTTGT | | |
| SNP_6 | chr2:32287318 | TTCTCTGGCAAGCAAACACATACA | A | scaffold139913 |
| | | CTTGATGATGCCTTGTTCAACTTT | | |
| | | TC[T/A]GCTATAAATGCAGCAAA | | |
| | | TTTCTTATTTTCACCTACTGTAGT | | |
| | | AYGCATTAK | | |
| SNP_7 | chr2:32287709 | TTMCAACATTATTTTTTWGATGAT | A | scaffold73333 |
| | | ACTACTGCTCAATATCTTATGAAA | | |
| | | TT[C/A]TCTTAAGAGACAAACAA | | |
| | | GGAGCAAAATGAAAAGATGAATCA | | |
| | | CCTGCTCAT | | |
| SNP_8 | chr2:32280848 | TGCAAATCAATGGTACAGTCAATG | C | scaffold139913 |
| | | TCTTCGACCACCAAGATGGACTTA | | |
| | | TT[A/C]GCCGTGGCCACAAGCAG | | |
| | | CCGTCTTAWATCAGAGTYTCTCAT | | |
| | | CACCTCAGT | | |
| SNP_9 | chr2:32284354 | AGCACAATTGCATCAGGATWTATC | A | scaffold73333 |
| | | CTATGAGTAGCATCTGCAATAGCT | | |
| | | TG[G/A]ACGCAAGTTACACTTTC | | |
| | | CTCCAATGAKACRGCTGTTTTTGC | | |
| | | ACCAATTGA | | |

### Markers Validation

The candidate markers were tested on various genotypes for validation and all markers behaved similarly. The 1441-16 donor line genotyping result was homozygote alternative. The negative controls selected, homozygote reference results were found, while other samples including progenies from 1441 crosses displayed Heterozygote result, indicating the presence of both known and novel Tm1 alleles (Table 5). All the 8 markers designed gave similar results demonstrating the difference indicating the homozygote reference, heterozygote and homozygote Alternative allele

TABLE 6

| | SNP ID | | | | | | | |
|---|---|---|---|---|---|---|---|---|
| | SNP1 | SNP2 | SNP3 | SNP5 | SNP6 | SNP7 | SNP8 | SNP9 |
| [symbol] ting allele | T | A | C | G | A | A | C | A |
| 4198 F1-146 | Het | Het | Het | Het | Het | Het | Het | Het |
| 4198 F1-146 | Het | Het | Het | Het | Het | Het | Het | Het |
| 4198 F1-149 | Het | Het | Het | Het | Het | Het | Het | Het |
| 4198 F1-149 | Het | Het | Het | Het | Het | Het | Het | Het |
| 4198 F1-3 | Het | Het | Het | Het | Het | Het | Het | Het |
| 4198 F1-3 | Het | Het | Het | Het | Het | Het | Het | Het |
| 4198 F1-4 | Het | Het | Het | Het | Het | Het | Het | Het |
| 4198 F1-4 | Het | Het | Het | Het | Het | Het | Het | Het |
| 4198 F1-5 | Het | Het | Het | Het | Het | Het | Het | Het |
| 4198 F1-5 | Het | Het | Het | Het | Het | Het | Het | Het |
| 4198 F1-6 | Het | Het | Het | Het | Het | Het | Het | Het |
| 4198 F1-6 | Het | Het | Het | Het | Het | Het | Het | Het |
| S. hab 3-5 | Het | Het | Het | Het | Het | Het | Het | Het |
| S. hab 3-5 | Het | Het | Het | Het | Het | Het | Het | Het |
| S. hab 3-5 | Het | Het | Het | Het | Het | Het | Het | Het |
| S. hab 3-5 | Het | Het | Het | Het | Het | Het | Het | Het |
| S. hab 3-3 | Het | Het | Het | Het | Het | Het | Het | Het |
| S. hab 3-3 | Het | Het | Het | Het | Het | Het | Het | Het |
| S. hab 3-3 | Het | Het | Het | Het | Het | Het | Het | Het |
| S. hab 3-3 | Het | Het | Het | Het | Het | Het | Het | Het |
| P1441-16 | Het | Het | Het | Het | Het | Het | Het | Het |
| P1441-16 | Het | Het | Het | Het | Het | Het | Het | Het |
| P96 | C:C | G:G | T:T | A:A | T:T | C:C | A:A | G:G |
| P96 | C:C | G:G | T:T | A:A | T:T | C:C | A:A | G:G |
| P98 | C:C | G:G | T:T | A:A | T:T | C:C | A:A | G:G |
| P98 | C:C | G:G | T:T | A:A | T:T | C:C | A:A | G:G |
| 815-1540 | Het | Het | Het | Het | Het | Het | Het | Het |
| 815-1540 | Het | Het | Het | Het | Het | Het | Het | Het |
| 815-1542 | Het | Het | Het | Het | Het | Het | Het | Het |
| 815-1542 | Het | Het | Het | Het | Het | Het | Het | Het |
| 816-1544 | Het | Het | Het | Het | Het | Het | Het | Het |
| 816-1544 | Het | Het | Het | Het | Het | Het | Het | Het |
| 816-1546 | Het | Het | Het | Het | Het | Het | Het | Het |
| 816-1546 | Het | Het | Het | Het | Het | Het | Het | Het |
| 816-1547 | Het | Het | Het | Het | Het | Het | Het | Het |
| 816-1547 | Het | Het | Het | Het | Het | Het | Het | Het |
| 817-1551 | Het | Het | Het | Het | Het | Het | Het | Het |
| 817-1551 | Het | Het | Het | Het | Het | Het | Het | Het |
| 817-1552 | Het | Het | Het | Het | Het | Het | Het | Het |
| 817-1552 | Het | Het | Het | Het | Het | Het | Het | Het |

[symbol]

indicates data missing or illegible when filed

The population 4198 F2 was generated through a cross between S. lycopersicum cultivated lines 98 and 1441, resulting in the hybrid named 4198 F1. F2 seeds were obtained through self-fertilization of the R1 plants. The 4198 F2 population consisted of 245 plants sown on Aug. 1, 2022, inoculated with ToBRFV and phenotyped for ToBRFV symptoms on September 13. The plants were subsequently divided into two groups: those exhibiting symptoms (148 plants) and those without symptoms (97 plants). The phenotyping and symptom evaluation procedures were the same as those described for the BC1F1 2222 population in addition, fruits phenotyping was conducted with the same DSI ladder (FIG. 3). The eight markers developed were used to Lest correlation to phenotype to allow use in breeding MAS process. The 41198 F2 population resulted in phenotypic segregation as follows on Table 7 below:

TABLE 7

| | (0) no | (1) light | (2) severe | (3) very severe |
|---|---|---|---|---|
| | visible | mosaic | mosaic | mosaic |
| | symptoms | pattern | pattern | pattern |
| Leaves mosaic | 8% | 31% | 48% | 13% |
| pattern: % | | | | |
| from the | | | | |
| population | | | | |
| ToBRFV | 20% | 47% | 27% | 3% |
| symptoms on | | | | |
| fruits % from | | | | |
| the population | | | | |

### Genotype—Phenotype Correlation of the Designed Markers

Following the markers validation described above, the 9841 F2 population was genotyped to all 8 markers. As observed on the validation plate, similar results were found across the markers, separating between the genotypes of homozygote alternative or reference and heterozygote. The population genotypic segregation results are like the expected of single mendelian gene in F2 population (p<0.0001). all the plants genotyped with homozygote reference results had severe symptoms (“2” and “3”), which indicates strong affinity of the marker to the resistance trait (Table 8). The distribution of phenotypes in the Homozygote alternative and heterozygote plants demonstrate that the new Tm1 allele contributes to the resistance trait. The presence of symptomatic plants in Homozygote alternative group, demonstrate the allele contribution to resistance is imperfect. However, the partial-dominance effect is clearly observed with the segregation of the heterozygote genotyped plants. and seems partial, as the enrichment of resistant plants with the overall phenotype-genotype correlation coefficient (R²) of 0.22.

TABLE 8

| | Symptoms level on leaves | | | | | |
|---|---|---|---|---|---|---|
| Genotype | 0 | 1 | 2 | 3 | Total | Total % |
| Homozygote | 18 | 20 | 13 | 0 | 51 | 22 |
| alternative | | | | | | |
| Heterozygote | 12 | 42 | 65 | 5 | 124 | 53 |
| Homozygote | 0 | 0 | 31 | 27 | 58 | 25 |
| reference | | | | | | |
| Total | 30 | 62 | 109 | 32 | 233 | 100 |

S. habrochaites 17-1-3-5 Source Genetic Background

### Mapping Population

The population BC1F1 2222 was generated through a backcross of a Solanum lycopersicum cultivated line 98 with S. habrochaites 17-1-3-5 (designated as 2209 F1; FIG. 15). 43 plants from the F1 cross were grown from March to August 2022 after inoculation with ToBRFV. One plant (238) displaying high resistance to the virus was selected for backcrossing with S. habrochaites 17-1-3-5, which was maintained as a clone through vegetative propagation. The resulting BC1F1 2222 population consisted of 286 plants sown on Aug. 1, 2022 and inoculated with ToBRFV on Aug. 14, 2022. On September 13, the young leaves of the plants were phenotyped for ToBRFV symptoms, and the plants were subsequently divided into two groups: those exhibiting symptoms (94 plants) and those without symptoms (194 plants). Weekly evaluations of symptoms were conducted using the disease severity index (DSI) as described by Zinger et al. (2021): (0) no visible symptoms; (1) light mosaic pattern on the apical leaf; (2) severe mosaic pattern on the apical leaf; (3) very severe mosaic pattern coupled with pronounced elongation or folding of the apical leaf. This population used for mapping.

The BC1F1 2222 population leaves phenotypic segregation collected 88 days after inoculation were as follows: 14% with “0”; 47% with “1”; 36% with “2”; and 3% with “3” (Table 9).

TABLE 9

| | (0) no | (1) light | (2) severe | (3) very severe |
|---|---|---|---|---|
| | visible | mosaic | mosaic | mosaic |
| | symptoms | pattern | pattern | pattern |
| % from the | 14% | 47% | 36% | 3% |
| population | | | | |

The population BC1F1 2223 is a sister population to BC1F1. The population was generated through a backcross of a Solanum lycopersicum cultivated line 98 with S. habrochaites 17-1-3-5 (designated as 2209 F1). 43 plants from the F1 cross were grown from March to August 2022 after inoculation with ToBRFV. One plant (393) displaying high resistance to the virus was selected for backcrossing with S. habrochaites 17-1-3-5, which was maintained as a clone through vegetative propagation. The resulting BC1 F1 2223 population consisted of 224 plants. This population was not used for mapping but displayed similar phenotypic results to BC1F1 2222.

### Data Pre-Processing

The reads of all parental lines (98, BC0F1-2209-238, 17-1-3-5) were aligned to the Heinz genome assembly (version SL4.0) as reference. The reference genome used in the pipeline was downloaded from solgenomics.net/organism/Solanum_lycopersicum/genome (version SL4.0).

### QTL Analysis

QTL analysis was performed using a regression model on each of the contigs using the values stored in the CGM. This regression is related to the wild sample inheritance patterns, as CGM represents the alleles originated from the wild sample only. QTL scan for the ToBRFV tolerance phenotype was conducted as follows. Table 10 describes the variance explained at each QTL independently. The joint model of the 3 loci at chr2 explains 27% of the variance.

TABLE 10

| Chr | Loci | Var. Explained % |
|---|---|---|
| 2 | ~18M | 13 |
| 2 | ~30M | 15.5 |
| 2 | ~36M | 11 |
| 1 | ~70M | 5 |

A QTL scan was performed by regressing the phenotype score on the genotype at each contig. A significant QTL was declared if a model including the genotype was substantially better than a model without the genotype using a likelihood-ratio test.

A logarithm Of Odds (LOD) score was calculated by comparing the variance explained by the two models as indicated in Broman et al. (Ch 4.1, Pg177). A Threshold of LOD>6 was used to, declare significant, results. Contigs that passed the LOD>3 threshold were subject to further examination and confidence intervals were calculated for the selected peaks based on 4.5-6.5 units in LOD scores from the most significant contig.

reference is now made to FIG. 16 depicting the LOD scores at each chromosome. The panels describe the different chromosomes based on the reference. Only reads that were successfully mapped to the reference genome are shown. A horizontal line indicating LOD=4.5 is indicated in black.

### OTL Results

SNP variants from the wild accession were found to be present in a significant proportion at the peak explaining up to 27% of the binary “Resistant/Susceptible” Phenotype variance. There is evidence in the LOD score curve that the source of this correlation is at the spanning most of Chromosome 2. The 95% confidence interval for the suggested region is at Chr2:0-35 [Mbp].

### Genotype/Phenotype Counts

The effect of genotype on the phenotype at the peak of the QTL loci is depicted in Table 11. The Genotypes column indicates the sample zygosity state. The columns Resistant and Susceptible denote the number of samples of each state in each group. The Chi2 p value column corresponds to the Chi² test for independence.

TABLE 11

| | | Chi2 p- | | | |
|---|---|---|---|---|---|
| Chr | Loci | value | Genotype | Resistant | Susceptible |
| 2 | ~18M | 6.1e−08 | G11 | 103 | 36 |
| | | | G10 | 21 | 30 |
| | | | G00 | 14 | 30 |
| 2 | ~29M | 1.3e−07 | G11 | 82 | 30 |
| | | | G10 | 15 | 17 |
| | | | G00 | 16 | 40 |
| 2 | ~36M | 4.7e−05 | G11 | 82 | 35 |
| | | | G10 | 23 | 17 |
| | | | G00 | 12 | 28 |

### Marker Sequences

Unique sequences that could be used as molecular markers for the selected regions were extracted using the following pipeline:
1. Extract K-mers of 1000 bp from the BC samples.
2. Filter for poor GC content (keep sequences with 30<GC<70).
3. Filter using BLAST against the cultivated reference (solgenomics.net/organism/Solanum_lycopersicum/_genome version SL4.0) by requiring an alignment proportion smaller than 40% within each K-mer.

1000 bp sequences markers derived from the wild accession sequences for each of the chromosomal loci that showed correlation with the phenotype were identified.

There are 59 sequences spanning over the regions of interest in Chromosome 2.

### Marker Selection:

Markers were selected using the VCF files of the population, such that they fall at the region of interest, their GC content is 35-65%, the flanking region is checked to be clean of IN/DELs and the sequences were re-blasted to the scaffold level assemblies of the 98, 17-1-3-5 parents in order to exclude multi mapped sequences. Non-informative variance were reported as uiapac abbreviations at the Flanking regions, while the informative variance was emphasized as an SNP variation at the corresponding column/s. SNPs were flanking region shown irregular coverage and/or unclear contribution to the variance explanation were excluded. The list of markers and sequences is specified in Table 12 below:

TABLE 12

| Marker ID | Chr | Position | Upstream_Sequence | Susceptible | Resistant | Downstream_Sequence |
|---|---|---|---|---|---|---|
| ToBRFV 2_2_1 | 2 | 7186620 | ATATCTTGTTCTGTTGATGATTGCTTCGAT | T | G | GTAATTCTTGACTACCCACCGCCCCCGGCT |
| ToBRFV 2_2_2 | 2 | 16019429 | TGAGTCCTCCTTGAGGGCTTCTTGCGGGGC | C | T | CTTGGGGAGTCGTACCTGGACGTTTTCAAT |
| ToBRFV 2_2_3 | 2 | 16148903 | AAAGCATTTGCAGCTTCCAAAAAGCTATGG | G | A | CAAGTGTTGTTAAGGAATCACACGAACATT |
| ToBRFV 2_2_4 | 2 | 19852516 | TCTCTTGAACGATGCTACGGTCCAAGTCTT | T | C | CTTACCAAATGCTACTMACCTACCAACAAC |
| ToBRFV 2_2_5 | 2 | 20133100 | AGAAGCAAACAACCATCTCTTTATCTTCAG | C | T | AGAAGMTGAATATAGAGCAGTCCGGAATGT |
| ToBRFV 2_2_6 | 2 | 22604243 | ATTGAGGTGATTTGTGTTTAGGAGTGACTC | C | T | CGGACGAATAACTTTCTTGGTTACAAAATA |
| ToBRFV 2_2_7 | 2 | 29236938 | GTTTTGAGCTTTCCCTTTTGAGAAAATGAC | T | G | AGAAGACCCATTGATTGGATTGGCAAGAAC |
| ToBRFV 2_2_8 | 2 | 29258772 | ATCATCAATTGGCATTCGTTTCCTTGCTGC | A | G | CACATGTGAACATTTCCCCAGTTGAAAAAA |
| ToBRFV 2_2_9 | 2 | 29259651 | TTTGGCCTGAGTACCATTTTCAGCTGGCAT | A | G | ACATAAGAGGGCTTATTGGGTTTACTATTA |
| ToBRFV 2_2_10 | 2 | 30451704 | TGTGTAGATAAATGCTTTTTCCAATAATTT | T | C | CCGMGATAAGGATACACAAGTCAGCAACCT |
| ToBRFV 2_2_11 | 2 | 30530325 | AGGAAGATTGAGCTAAAGAAAATAACAYAT | C | T | TCACTACCTGACACCAACAACTACCTTGAA |
| ToBRFV 2_2_12 | 2 | 30560729 | AACATATTCCTTCCTTTTCTAACCCCGCAA | G | A | TTCTGCAAGCTTGTAGTTTTTGTAGRTTGT |
| ToBRFV 2_2_13 | 2 | 30562593 | AGAGGTTTTTCGATAGACCTTCSGCTCAAA | T | C | GAAAAGTTTTCAAAGCAGGATTGTAAGAAA |
| ToBRFV 2_2_14 | 2 | 30633731 | AACTGAGACTGAATTGTGCCCGAACATAGC | G | A | GAAKTGGCATAGAAAATTCATACAGCTGAC |
| ToBRFV 2_2_15 | 2 | 30763962 | GGTAGGATTTASAGCGACAACGATCATAAA | G | T | TTAAAGTTTCTGGTCTCCTGTTTGGCATTC |
| ToBRFV 2_2_16 | 2 | 30775549 | ATTATAGCGACTTCCCTCATCTTTATTGGC | G | A | AGTTGATAGTGAAGGAAAATGAGTGMGAAA |
| ToBRFV 2_2_17 | 2 | 30787440 | CCTTCACTATAAGTCCTAAASTCTTGCAAA | A | C | TTTTCCTAAGCATTTCTCACGACTGGCTGT |
| ToBRFV 2_2_18 | 2 | 30789154 | TATCAAGGAAAAAAWTGTCCTCTGATACAT | A | C | GAAAGTGGTTTGATTGCCGCGTATGTAAAA |
| ToBRFV 2_2_19 | 2 | 30790287 | ATACTWGGTGGAGCAAGTAACAAGCAACGG | C | A | ATTACTGTCTTTAAAATAGACTCTGTATTG |
| ToBRFV 2_2_20 | 2 | 30793033 | GTGAAACTCTCTTCTGAAGGTTKATCTATC | A | T | CACTTATGCTCTTGCAAATACCTCTACAAG |
| ToBRFV 2_2_21 | 2 | 31279480 | AAAAGTACAGCTGACAGATTCCCCAATCCT | G | A | YGACATCCACACATTTTCTACCAGTTAAAG |
| ToBRFV 2_2_22 | 2 | 31296546 | GCGATTTTCGCTTGAGAAAAGACTTATCCA | T | A | GTCATTATTYGTTAACTGAAAACAACTTCG |
| ToBRFV 2_2_23 | 2 | 31311682 | AGGGGATTCCATATACAAATTTTTATACGG | G | A | AGTCAGGCTCGTTTTTTTAGYGATGAGATT |
| ToBRFV 2_2_24 | 2 | 31348408 | TTCAAAAATAGCATTCCTCAAAACTTCTCC | G | A | GGCTGAAYGTTGAGAAGTCAGGAGATTATG |
| ToBRFV 2_2_25 | 2 | 31499338 | TAATGTTTGGGCACAKACAGAAGGTTTGTG | T | C | ATCCCACACACCCCATGTAAGTCCGTGTAT |
| ToBRFV 2_2_26 | 2 | 31509218 | GGTGGAACACTTGCAGAGTTGCATGATGTT | G | T | CCCTCTTTCTTCGGCAGGTYCATGATATGA |
| ToBRFV 2_2_27 | 2 | 31564251 | TGATAGGGATGGCCTTGTGTTTTGTTCTTC | T | A | TTCATTCGTTTGTTCYATTACAAAGGGCAT |
| ToBRFV 2_2_28 | 2 | 31640577 | GCAAACTADCTTGGAATCTAATGGCCCTAG | C | A | AACCTCCAGAATCTGTCTGTGTCTCCTCTC |
| ToBRFV 2_2_29 | 2 | 32284107 | ACACGCCAAGGAATTAAGACAAAATSACTA | A | C | AAAATTAATGAAGCCGGTGAACTCATCTGG |
| ToBRFV 2_2_30 | 2 | 32323793 | CAAAAGTACAGCTGATCCAACCTCAAACAA | T | C | CCCCTGRTACTTGGAATATATGATAATTAG |
| ToBRFV 2_2_31 | 2 | 32372603 | TTTTTTTCTTCGGTCGGATCCCTCTTCCAC | T | C | TTTAAGAATATTACATCTCAAAMATGTGGT |
| ToBRFV 2_2_32 | 2 | 32408841 | TTGTGCTCAGATTTAGCCGCTWGCTTATGC | T | G | AAAGAAAGAGATTGAAGTCCACAGTTTTGC |
| ToBRFV 2_2_33 | 2 | 33254552 | CCTTCCTGACTTGTCCGATGAGCAATTGCT | T | C | ARSGAAATTGAGTATCTTTTGAAAAATGGA |
| ToBRFV 2_2_34 | 2 | 33268238 | GAAAGAGTTCAAGCTCATTTAGCTTGACAA | A | G | TAMTATAGAAAAGGCAACCTTTTCTTAGGC |
| ToBRFV 2_2_35 | 2 | 35719949 | GAGGGTGTTCTGAGGTTAGCATTTAATTTG | T | C | AACAGCCCTTTCTGSAACATATGCTAAATT |
| ToBRFV 2_2_36 | 2 | 36216379 | TACTCGATAGCTTCGGTTGTTATGAATTTG | C | A | TAAMTTCTTAGGTTCAGAGGTTCAGTCTAT |
| ToBRFV 2_2_37 | 2 | 36237338 | GGAGGATTCATGAAATAACTAACTCRAGAG | A | T | ATCTCCAATTACAAAAATCTCACTGGGATA |
| ToBRFV 2_2_38 | 2 | 36250710 | TCCTACTAGTTATTCCTGATCAAAACTCCT | T | C | TTTGTGGGTGMATCTACTTGGTCTTACCAG |
| ToBRFV 2_2_39 | 2 | 36322997 | TTCAAAATTGATGTYGGAATGGGTTCCTGT | C | G | AAGATGCAAGTTGTTTTTAGTCTGCAAGTG |
| ToBRFV 2_2_40 | 2 | 36358580 | TYATTTTGGCAGAGTATGTGAATCTATTTC | G | A | TTTGCCACATTTCATCTCAAAGTAAACGGG |
| ToBRFV 2_2_41 | 2 | 36386306 | ACCTGAGCAGGAGCAGGCACGAATCTGAAA | T | C | CACATAGRTCAATTATCTTTTTGTCAACAC |
| ToBRFV 2 2 42 | 2 | 36388770 | GAACTAAGAAGAAGAAGAATACAGGGAGGT | A | C | CAAAAAATATACACCAAGGGGTCKTTAGAG |
| ToBRFV 2_2_43 | 2 | 36405731 | CTCCTTACCTCCAAGAGAGACGGATAAAAT | C | A | TGCATAYACTTTATCCTCCCAAAGCCTCAC |
| ToBRFV 2_2_44 | 2 | 36437145 | GTGTCCTTTCGTACCATCATCCTCGTCGTC | T | G | TCWTCATCATCATAAGATGCAAGTGGAAGA |
| ToBRFV 2_2_45 | 2 | 36438952 | TAGTGGCGGAGCCAGGCTGCAAAACASTTA | T | C | TAGGAACCCTCTAACCCTACAACCAGAAAT |
| ToBRFV 2_2_46 | 2 | 36445713 | GAAAGAATGCCTTCTAAAAACTGTGCATCC | A | G | TCTCACCARAAATACCAATCACCCCAAGTA |
| ToBRFV 2_2_47 | 2 | 36447457 | GTTTTACCAGTACATAGCCAATGCACAGCT | T | A | TACAWAGCACAGGGCAATATATTTAGAGAA |
| ToBRFV 2_2_48 | 2 | 36460455 | AAAGATGAGAGGGATTCTCCGGCGGTTGAT | C | A | GGCGGCAGACAGCCATTYACATCATCACGG |
| ToBRFV 2_2_49 | 2 | 36463543 | GYGCTAGTTATGTAAGGGTGTCATTTAGTA | C | T | GCTGGACACACATAGATAGTCTTGGGATAA |
| ToBRFV 2_2_50 | 2 | 36581002 | ATAATCACGAGCAACSACAAGTAAAAACCC | T | C | AGATTTCCTGCTTTTGCAGCAGAGTGGCCC |
| ToBRFV 2_2_51 | 2 | 36602821 | AAACAAGGTGGTCTAYTACTCGAGGAGCTC | T | A | GTGCGGAGTGTGAAACAAGTGCAAAGGATA |
| ToBRFV 2_2_52 | 2 | 36788835 | ATCTAGACAYCCCAAGTTGAAATTGCCGAT | T | A | GATTCTGAGTGTTTGACTCAAGAACAGGCT |
| ToBRFV 2_2_53 | 2 | 36789963 | GCTGAAGAAAGGTATGCWAATTGGAATTTC | G | A | CCAGATTTAGTGACTTACACCATTCTAATG |
| ToBRFV 2_2_54 | 2 | 36791094 | CACAACAGCAAGAATTTAGAAAAGCCTATA | A | T | TGAAAAGATMCTTCCACCATCCATCATGTG |
| ToBRFV 2_2_55 | 2 | 36799921 | TTCGGAATCATTCCMCAGGGTCCAAATATC | G | A | ACTATTCCTCTCCACATGATCGAGAGAAGT |
| ToBRFV 2_2_56 | 2 | 36997642 | CTTGTTAAGCTCCTTCGACATAGAGAACTT | G | T | CTGGTGCATTTAGTGAATTTAGAAGCRTAC |
| ToBRFV 2_2_57 | 2 | 37141529 | AAATTGCTTAGTCATGTGATATTCGCATTC | A | G | TCAGCAATGACATGCCACCATCTTYCCCTT |
| ToBRFV 2_2_58 | 2 | 38393498 | ATAACTCGATYTCACACCCACAGTAGTTAA | C | T | GTATGGGGAAGGAATACATTAAGTTTCATG |
| ToBRFV 2_2_59 | 2 | 38421004 | GAAACTCTTTCAARACAGCATCTTTGGAAT | G | A | TGAAAGCAGAGCCCTTGTGGTACATTTTCT |

1-13. (canceled)

14. A cultivated tomato plant harboring a partially dominant heterologous resistance gene or genes to tomato brown rugose fruit virus (ToBRFV), wherein the resistance gene or genes form part of a QTL derived from chromosome 7 of a ToBRFV-resistant tomato plant.

15. The cultivated tomato plant of claim 14, wherein the QTL comprises at least one marker located on the ToBRFV-resistant tomato plant between 62 Mbp to 65 Mbp.

16. The cultivated tomato plant of claim 15, wherein the at least one marker is selected from the markers listed in Table 3.

17. The cultivated plant of claim 14, wherein said plant further comprises at least one additional ToBRFV-resistance conferring QTL derived from chromosome 2 of the ToBRFV-resistant tomato plant.

18. The cultivated plant of claim 17, wherein the QTL comprises at least one marker located between 0 Mpb to 43 Mbp.

19. The cultivated tomato plant of claim 14, wherein said plant is the offspring of crossing a ToBRFV-resistant wild tomato plant comprising the QTL on chromosome 7 with a ToBRFV-susceptible cultivated tomato plant or of a ToBRFV-resistant wild tomato plant comprising the QTL on chromosome 7 with a ToBRFV-susceptible wild tomato plant.

20. The cultivated tomato plant of claim 14, wherein the ToBRFV-resistant tomato plant is a wild tomato plant selected from a group consisting of LA0107 (S. corneliomulleri), LA0361, LA1918, LA2650 (S. habrochaites), LA1938, LA1969, LA2748, LA2755 and LA2931 (S. chilense).

21. The cultivated tomato plant of claim 14, wherein the ToBRFV-cultivated tomato plant is Solanum Lycopersicum.

22. A seed of the cultivated tomato plant of claim 14.

23. Propagation materials of the cultivated tomato plant of claim 14.

24. A genetic marker for identification or selection of tomato plants harboring partially dominant resistance to tomato brown rugose fruit virus (ToBRFV), wherein the genetic marker is selected from the markers listed in Table 3.

25. The tomato plant of claim 14, wherein said plant is the offspring of crossing ToBRFV-resistant cultivated tomato plant source 1441 with a hybrid ToBRFV resistant cultivated tomato plant.

26. A seed of the tomato plant of claim 14, wherein said plant is the offspring of crossing ToBRFV-resistant cultivated tomato plant source 1441 with a hybrid ToBRFV-resistant cultivated tomato plant.

27. Propagation materials of the tomato plant of claim 14, wherein said plant is the offspring of crossing ToBRFV-resistant cultivated tomato plant source 1441 with a hybrid ToBRFV-resistant cultivated tomato plant.

28. A cultivated tomato plant comprising in its genome an introgressed locus that confers resistance to ToBRFV, wherein a representative sample of seeds of the plant has been deposited under accession number NCIMB 43817.

29. A method of identifying a ToBRFV-resistant tomato plant comprising the step of genotyping a population of tomato plants and selecting plants having at least one genetic marker of claim 24.
